(19) United States
(12) Patent Application Publication
(10) Pub. No.: US 2006/0200127 A1
(43) Pub. Date: Sep. 7, 2006

(54) Method and apparatus for external fixation of bone fractures

(76) Inventor: Wardak Mohd Ismail, Kabul (AF)

Correspondence Address: LITMAN LAW OFFICES, LTD, PO BOX 15035, CRYSTAL CITY STATION, ARLINGTON, VA, 22215, US

(21) Appl. No.: 11/062,430

(22) Filed: Feb. 23, 2005

(51) Int. Cl. A61B 17/60

(52) U.S. Cl. 606/59

(57) ABSTRACT

The method and apparatus for external fixation of bone fractures provides for external fixation of bone fractures, especially fractures of the patella and the olecranon. An apparatus for external fixation of bone fractures comprises a support member, and a pair of tensioning assemblies disposed along the support member. A first tensioning assembly is connected to the first end of the support member, and a second tensioning assembly is slidably disposed along the support member. Filaments, attached to and extending between the first and second tensioning assemblies, may be tensioned by moving the second tensioning assembly along the support member away from the first tensioning assembly. Filaments, such as Kirschner wires, are inserted through bone fragments on opposite sides of a fracture. The filaments are tensioned by moving the tensioning assemblies apart. Tension applied to the filaments draws the filaments together, compressing the bone fragments together at the fracture.

### BACKGROUND OF THE INVENTION

1. Field of the Invention

The present invention relates to treatment of orthopedic fractures, and more particularly to a method and apparatus for external fixation of bone fractures, especially fractures of the patella and the olecranon.

2. Description of the Related Art

Various methods are employed to set a broken bone and to maintain bone fragments in a fixed position during a period of healing. Relative movement of the bone fragments must be prevented during the healing period so that the bone may properly heal. Additionally, it may be desirable to keep the bone fragments under compression during healing, because compression of the bone fragments increases the rate of healing.

A cast, or splinting, is often adequate for fixation of some types of fracture. However, certain bones, or portions of certain bones, are not adequately stabilized or fixated by casting or splinting. Surgical techniques may be employed to place pins, wires, screws, plates, and the like to hold bone fragments in place. Various techniques for placement of the pins, wires, screws, and plates have been devised, and frequently require an open surgical technique to place pins, wires, screws, or plates in fragments of the fractured bone, as well as in adjacent bones in some circumstances. Such open surgical techniques are often more invasive that is desirable. Additionally, further devices that may be employed to hold the pins, wires, screws, or plates stably in place can limit movement of an affected limb.

In certain bones, such as the patella and the olecranon, difficulty in treating a fracture is compounded because muscles in connection with the bones, or with fragments of the bones, tend to pull the bone fragments apart along a fracture, rather than compress the fragments together. Moreover, given their proximity and relation to limb joints (knee and elbow) the treatment of fractures of the patella and the olecranon may impact the motility of a patient if such treatment restricts the joint movement.

A minimally invasive technique for fixation of fractured bones, such as the patella and the olecranon, that allows early movement of knee and elbow joints with full weight bearing following a fracture and treatment, is desirable to promote a swift and full recovery.

Thus, a method and apparatus for external fixation of bone fractures solving the aforementioned problems is desired.

### SUMMARY OF THE INVENTION

The method and apparatus for external fixation of bone fractures provides for external fixation of bone fractures, especially fractures of the patella and the olecranon. An apparatus for external fixation of bone fractures comprises a support member, the support member being an elongated bar or rod having first and second ends. A pair of tensioning assemblies are disposed along the support member. A first tensioning assembly is connected to the first end of the support member, and a second tensioning assembly is slidably disposed along the support member. Each of the tensioning assemblies is adapted to retain a plurality of filaments extending between the first and second tensioning assemblies, such that the filaments may be tensioned by moving the second tensioning assembly along the support member away from the first tensioning assembly.

In a method for external fixation of bone fractures, using an apparatus for external fixation of bone fractures of the present invention, a fractured bone such as a fractured patella or olecranon is first reduced and temporarily fixated using conventional techniques. With the fracture reduced and temporarily fixated, filaments, such as Kirschner wires, are inserted through the bone fragments on each side of the fracture. The filaments are fastened between the first and second tensioning assemblies of the apparatus for external fixation of bone fractures, and the filaments then tensioned by moving the second tensioning assembly along the support member away from the first tensioning assembly, into a tensioned position. The second tensioning assembly is then fixed in the tensioned position.

The tension applied to the filaments tends to draw together the filaments on opposite sides of the fracture, pressing the bone fragments together at the fracture. Thus, the bone fragments are held securely together for healing.

These and other features of the present invention will become readily apparent upon further review of the following specification and drawings.

### BRIEF DESCRIPTION OF THE DRAWINGS

FIG. 1 is an environmental, perspective view of an apparatus for external fixation of bone fractures according to the present invention, placed to fixate a fractured patella.

FIG. 2 is a top view of an apparatus for external fixation of bone fractures according to the present invention, placed to fixate a fractured olecranon.

FIG. 3 is a side view of the apparatus for external fixation of bone fractures shown in FIG. 2.

FIG. 4 is a side view of a fractured olecranon, prior to reduction and fixation with the apparatus for external fixation of bone fractures according to the present invention.

FIG. 5 is a perspective view of the fractured olecranon having the fracture reduced, and K-wires placed for fixation.

FIG. 6 is a perspective view of the fractured olecranon showing the fracture reduced and fixated by the apparatus for external fixation of bone fractures according to the present invention.

Similar reference characters denote corresponding features consistently throughout the attached drawings.

### DETAILED DESCRIPTION OF THE PREFERRED EMBODIMENT

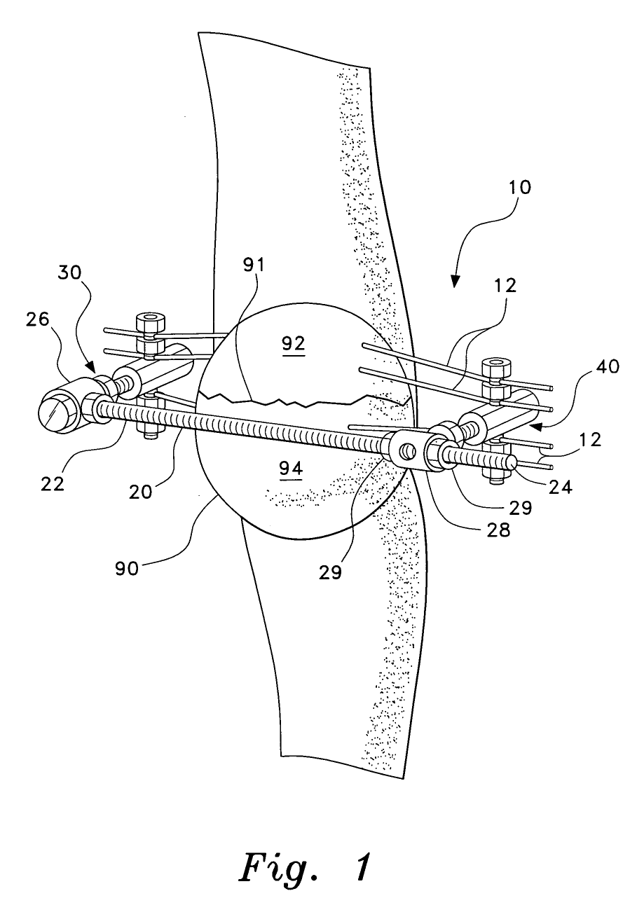

The present invention is a method and apparatus for external fixation of bone fractures, the method and apparatus being especially suited for external fixation of fractures of the patella and the olecranon. Referring to FIG. 1, an apparatus for external fixation of bone fractures, generally referred to as 10 in the figures, is shown employed in the fixation of a fractured patella 90.

The apparatus for external fixation of bone fractures 10 comprises a support member 20, which is an elongated rod or bar, in the illustrated embodiment, having a first end 22 and a second end 24. A pair of tensioning assemblies 30, 40 are disposed along the support member 20. A first tensioning assembly 30 is connected to the first end of the support member 20, and a second tensioning assembly 40 is slidably disposed along the support member 20. Each of the tensioning assemblies is adapted to retain a plurality of filaments 12 extending between the tensioning assemblies 30, 40, such that the filaments 12 may be tensioned by moving the second tensioning assembly 40 along the support member 20 away from the first tensioning assembly 30. A collar 26 holds the first tensioning assembly 30 in position at the first end 22 of the support member 20, while a collar 28 holds the second tensioning assembly 40 slidably along the support member 20. In the illustrated embodiment, a pair of locking nuts 29, disposed on opposite sides of the collar 28, may be used to position the second tensioning assembly 40 and to lock the second tensioning assembly 40 in place.

The apparatus for external fixation of bone fractures 10 is shown in FIG. 1 placed for the fixation of a fractured patella 90. A fracture line 91 in the patella 90 divides the patella 90 into a proximal fragment 92 and a distal fragment 94. A first pair of filaments 12 are passed through the proximal fragment 92, and a second pair of filaments are passed through the distal fragment 94. The filaments 12 are generally parallel to one another, and are generally parallel to the fracture 91, or transverse to the patella 90 or to the knee or leg generally. The filaments 12 may be Kirschner wires, as typically employed in orthopedic procedures, or may be other filamentous materials such as nylon, Teflon, other metal wires, and the like.

The filaments 12 are fastened between the first and second tensioning assemblies 30, 40, the ends of the filaments 12 being drawn slightly together in relation to their positions within the patella 90. It can be recognized that tensioning the filaments, by moving the second tensioning assembly 40 away from the first tensioning assembly 30, will draw the filaments 12 passed through the proximal fragment 92, and the filaments passed through the distal fragment 94 together, thereby pressing the proximal fragment 92 and the distal fragment 94 together at the fracture 91.

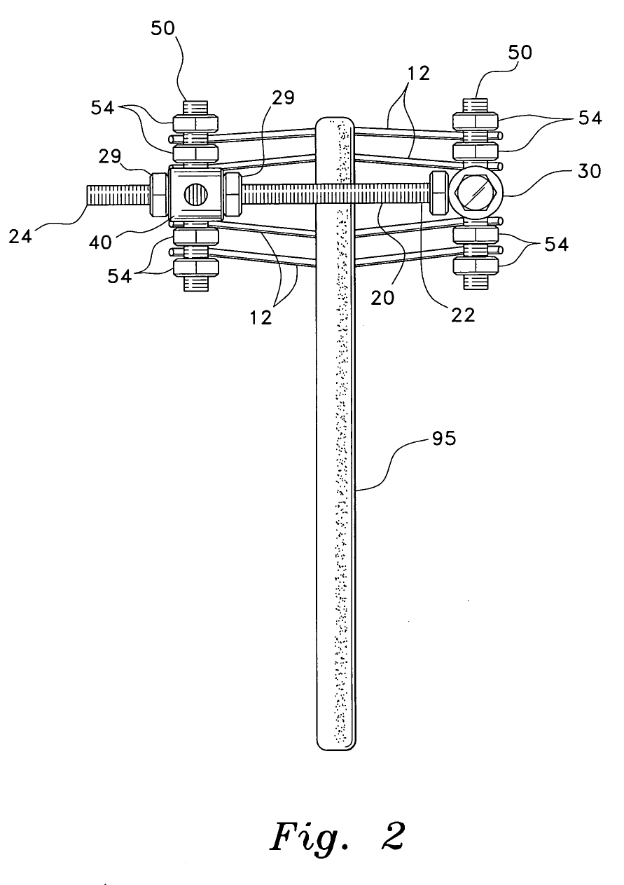

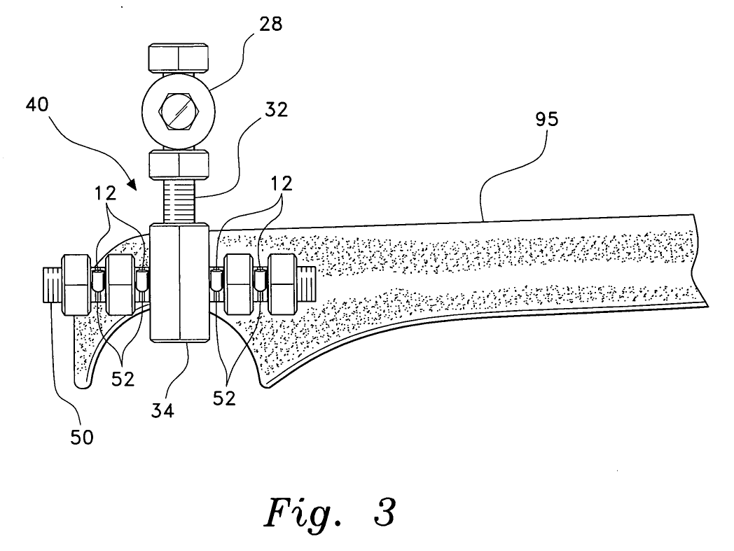

Turning now to FIGS. 2 and 3, an apparatus for external fixation of bone fractures 10 is shown in greater detail, employed in the fixation of a fractured olecranon 95. In the instant embodiment, each tensioning assembly 30, 40 comprises a post extending normal from the support member 20, the post comprising a threaded portion 32 and a spacer portion 34. Transversely attached to the spacer portion 34 is a filament holder 50. In the illustrated embodiment, the filament holder 50 is a threaded rod inserted through the spacer 34. Alternatively, a filament holder 50 may comprise a pair of short threaded rods each extending from opposite sides of the spacer 34. A plurality of apertures 52 are defined diametrically through the filament holder 50, the apertures 52 being spaced apart along the length of the filament holder 50. Clamping nuts 54 are disposed along the filament holder 50, and are used to clamp in place a filament 12 inserted through an aperture 52.

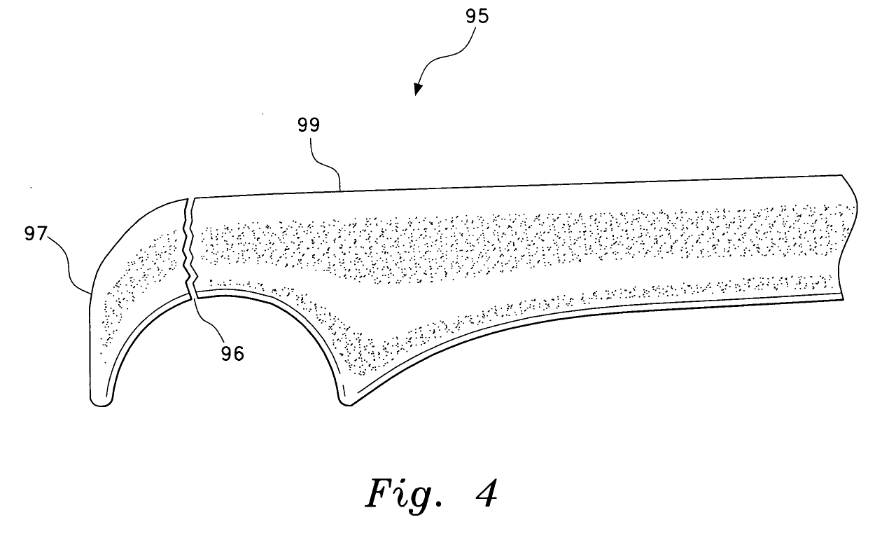

Turning now to FIGS. 4-6, a method for external fixation of bone fractures, employing an apparatus for external fixation of bone fractures 10 of the present invention, is described with reference to a fracture of the olecranon 95. It can be understood that a similar method may be employed for the external fixation of other bone fractures. Referring to FIG. 4, a fracture 96 of the olecranon 95, near the olecranon process, separates the olecranon into a proximal fragment 97 and a distal portion 99. Initially, the fracture must be reduced, bringing the proximal fragment 97 and the distal portion 99 together and into contact along the fracture 96. A closed method may be employed for the reduction of the fracture 96 if appropriate. Alternatively, an open procedure may be employed for proper reduction of the fracture 96 if required, prior to fixation of the fracture using a closed technique for placing the apparatus for external fixation of bone fractures 10.

Upon reduction of the fracture 96, percutaneous clamps may be employed for temporary fixation of the fragments 97, 99. An x-ray may be taken, prior to fixation using the apparatus for external fixation of bone fractures 10, to ensure adequate reduction.

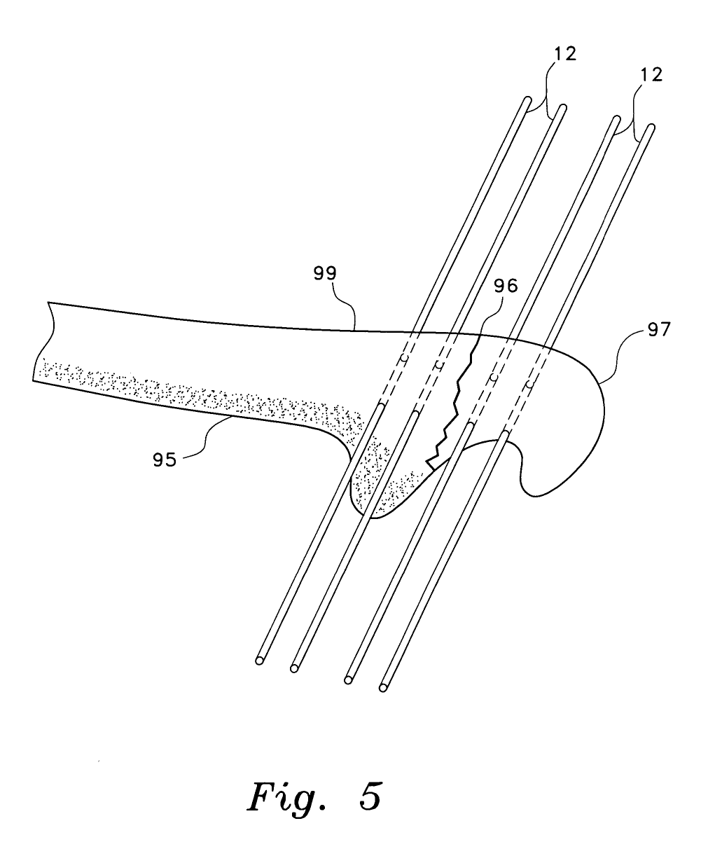

Turning now to FIG. 5, a first pair of filaments 12 is passed through the proximal fragment 97, and a second pair of filaments is passed through the distal portion 99. The first and second pairs of filaments 12 are generally parallel to one another, and located on opposite sides of the fracture 96. The filaments 12 extend generally transversely to the olecranon 95.

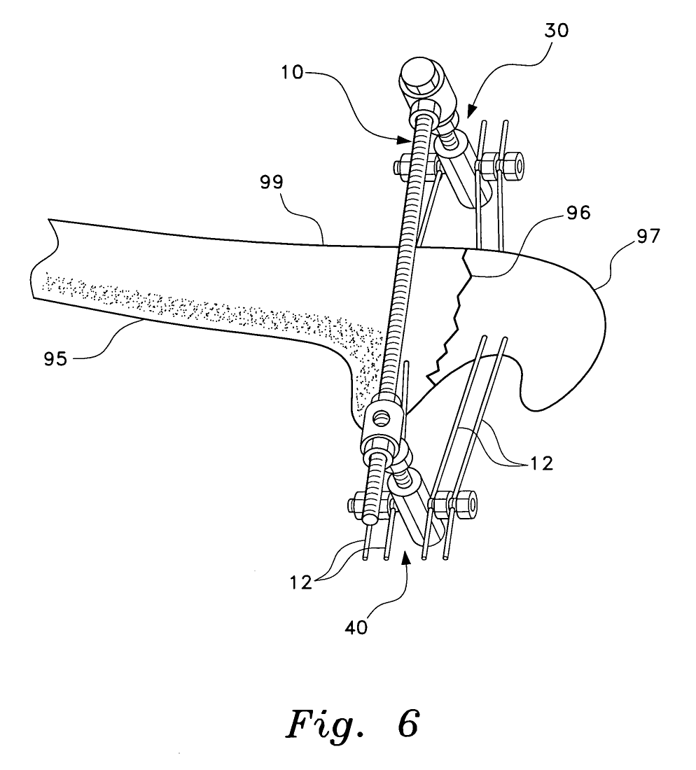

Turning now to FIG. 6, the filaments 12 are fastened between the first tensioning assembly 30 and the second tensioning assembly 40 of the apparatus for external fixation of bone fractures 10. Note that the ends of the filaments 12 are drawn slightly together in relation to their positions passed through the olecranon 95. The filaments 12 are then tensioned by moving the second tensioning assembly 40 away from the first tensioning assembly 30. It can be recognized that tensioning the filaments 12, will draw the filaments 12 passed through the proximal fragment 97 and the filaments passed through the distal fragment 99 together, thereby compressing the proximal fragment 97 and the distal portion 99 together at the fracture 96.

Because the apparatus for external fixation of bone fractures 10 is supported only by the fragments of the bone under treatment, and surrounding soft tissues, the apparatus for external fixation of bone fractures 10 may be used for treatment of fractures of the patella and the olecranon without negative impact to the affected joint. Thus, full movement of elbow or knee joints may be performed early following treatment, promoting rapid healing and rehabilitation.

It is to be understood that the present invention is not limited to the embodiment described above, but encompasses any and all embodiments within the scope of the following claims.

I claim:

1. An apparatus for external fixation of bone fractures, comprising:
a support member having first and second ends;
a first tensioning assembly connected to the first end of said support member;
a second tensioning assembly slidably disposed along said support member;
means for adjusting and fixing the position of said second tensioning assembly along said support member; and
a plurality of filaments extending between said first and second tensioning assemblies, the filaments being adapted for passage through a bone on opposite sides of a bone fracture.

2. The apparatus for external fixation of bone fractures according to claim 1, wherein each said tensioning assembly comprises a post extending normal to said support member, and a filament holding member transversely attached to said post.

3. The apparatus for external fixation of bone fractures according to claim 2, wherein each said filament holding member comprises at least one rod having a plurality of apertures defined diametrically through and spaced apart along the rod.

4. The apparatus for external fixation of bone fractures according to claim 1, wherein said filaments are wires.

5. The apparatus for external fixation of bone fractures according to claim 1, wherein said filaments are Kirschner wires.

6. An apparatus for external fixation of bone fractures, comprising:
a rod having first and second ends;
a first post connected to the first end of said rod, the post extending normal to said rod;
a first filament holding member transversely attached to said first post;
a second post slidably disposed along said rod;
a second filament holding member transversely attached to said second post;
means for adjusting and fixing the position of said second post along said support member; and
a plurality of filaments extending between said first tensioning assembly and said second tensioning assembly, the filaments being adapted for passage through a bone on opposite sides of a bone fracture.

7. The apparatus for external fixation of bone fractures according to claim 6, wherein said filaments are wires.

8. The apparatus for external fixation of bone fractures according to claim 6, wherein said filaments are Kirschner wires.

9. A method for external fixation of a bone fracture, comprising the steps of:
providing an apparatus for external fixation of bone fractures, the apparatus for external fixation of bone fractures comprising a support member having first and second ends, a first filament holding assembly connected to the first end, a second filament holding assembly slidably disposed along the support member;
reducing the fracture;
inserting at least one filament through a bone fragment on a proximal side of the fracture;
inserting at least one filament through a bone fragment on a distal side of the fracture;
fastening said filaments between said first filament holding assembly and said second filament holding assembly;
adjusting said second filament holding assembly into a tensioning position to tension said wires; and
fixing said second filament holding assembly in said tensioning position.

10. The method for external fixation of a bone fracture according to claim 1, wherein the step of reducing the fracture further comprises the step of temporarily fixating the fracture.

11. The apparatus for external fixation of bone fractures according to claim 9, wherein said filaments are wires.

12. The apparatus for external fixation of bone fractures according to claim 9, wherein said filaments are Kirschner wires.
